(19) United States
(12) Patent Application Publication
(10) Pub. No.: US 2004/0241666 A1
(43) Pub. Date: Dec. 2, 2004

(54) Ligand array assays that include an organic fluid wash step and compositions for practicing the same

(76) Inventors: Douglas A. Amorese, Los Altos, CA (US); Eric M. Leproust, San Jose, CA (US); Winny W. Ke, San Jose, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/452,767

(22) Filed: May 30, 2003

(51) Int. Cl. C12Q 1/68; C12M 1/34

(52) U.S. Cl. 435/6; 435/287.2

(57) ABSTRACT

Ligand array assays and compositions for use in practicing the same are provided. A feature of the subject methods is that they include a wash step in which the ligand displaying surface of a sample exposed ligand array is washed with organic wash fluid, e.g., propylene carbonate. Also provided are kits for use in practicing the subject methods. The subject methods and kits find use in a variety of ligand array based applications, including genomic and proteomic applications.

### FIELD OF THE INVENTION

The present invention relates to ligand, and particularly, biopolymeric arrays.

### BACKGROUND OF THE INVENTION

Array assays between surface bound binding agents or probes and target molecules in solution may be used to detect the presence of particular analytes in the solution. The surface-bound probes may be nucleic acids (e.g., oligonucleotides, polynucleotides), peptides (e.g., polypeptides, proteins, antibodies) or other molecules capable of binding with target biomolecules in the solution (e.g., nucleic acids, proteins, etc.). Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in discrete locations on a surface of a substrate (collectively referred to herein as an “array”) such as a glass substrate or the like. A solution containing target molecules (“targets”) that bind with the-attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate, which pattern is then detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

In the above-described assays, typically one or more wash steps and then a dry step are performed between the sample contact and array reading steps. In the one or more wash steps, the substrate surface of the array is typically washed with an aqueous fluid in order to remove unbound targets and other reagents from the substrate surface.

A number of different protocols have been developed for drying the substrate surface of an array following the one or more wash steps, so that the array may be read, e.g., scanned.

One of the most common methods currently employed uses an air or nitrogen knife to physically displace the wash solution left on the array following the wash step. If the array is placed in an automated or semi-automated hyb/wash station, e.g., placed in a chamber, a variation of this method uses gravity or pressure to empty the chamber, and then circulates a gas (usually air or nitrogen) through the chamber using an inlet and an outlet. The gas may be heated to increase the drying rate.

In another type of drying method, array slides are spun in a low to moderate speed centrifuge. With this method, the inertia generated by the centrifugical acceleration displaces the wash solution remaining on the array surface.

In yet another method, a squeegee made of flexible and inert plastic, such as silicone is employed. In this type of method, the wash solution remaining on the array surface is mechanically displaced by contact of a plastic lip on the array surface, followed by a lateral movement of the lip across the length of the array.

Finally, evaporation may be used to dry arrays. In this-method, the wash solution remaining on the array surface is simply removed by evaporation using a normal or dry atmosphere, and/or using an inert gas (such as nitrogen).

Each individual step in a given array assay protocol has a direct impact on the quality of data obtained from the assay, and therefore each individual step must be carefully controlled. For instance, the composition of the various buffers and their temperature, e.g., the solution stringencies of hybridization and washes, may impact both the hybridization of the probe sequence with its complementary sequences (perfect match=sensitivity) and with analog sequences or binding motifs (mismatches, non-Watson crick, etc.=specificity). Similarly, the drying process must be controlled to minimize the variations in the background and feature signals obtained, thus maximizing the signal/noise (S/N) ratio of the system.

The present inventors have now determined that the foregoing drying methods can produce various problems as will now be discussed. Some problems associated with various drying protocols currently employed in array processing, which can result in data of lower quality, are:

Local non-uniformity of background or feature signals. 1) During drying of individual features, temperature and/or salt concentration gradients due to evaporation may occur. They may result in denaturation of the duplex formed during hybridization, followed by non-uniform redistribution of the signal within the feature or its background (for instance, cometing, non-uniform feature morphologies, etc.). 2) Alternatively, salts present in the wash buffer may precipitate upon partial or complete evaporation of the aqueous media. Those salt particles will cover individual features only partially, and will create signal non-uniformity by affecting the quantum yield of the dyes in close proximity with them. 3) Alternatively, salt particles described in 2) may affect the optimum operation of the scanner, such as by reflecting part of the excitation or emitted light, or by perturbing the compensation performed by the autofocus.

Global non-uniformity. The problems described in the local uniformity sections are also applicable globally, i.e. at the array scale. Non-uniform drying, such as gradients in evaporation rates across the array, may result in gradients in the extent of denaturation or in the extent of particle formation. Therefore, features of the same sequence at different location across the array may report different background or feature signals due to drying artifacts.

Non-reproducibility/non-repeatability from array to array. Because the drying process is usually non-automated, the reproducibility of the drying and the extent of drying artifacts usually vary from array to array. Furthermore, even for automated drying processes, the local and global uniformity problem may also be variable because of the intrinsic variations introduced during evaporation of the aqueous media (temperature and concentration gradients) and precipitation of the dissolved reagents.

The air knife drying method (summarized above) suffers from all three of the above-described drying problems (i.e., local uniformity, global uniformity and reproducibility). Some reasons for these problems include: 1) the lack of control on the angle of the air stream with respect to the substrate from array to array and within an array; 2) the flow rate of the air knife gas varies from array to array and within an array; and 3) the increase in salt concentration due to evaporation as the sheet of buffer is displaced across the array. FIG. 1 illustrates such a non-uniform distribution of a green reporter dye after drying of a solution of this dye using a nitrogen gun (only the right half of the slide was wetted and dried to provide a reference).

The centrifuge method also suffers from the previously described drying problems. When placed in the centrifuge, momentum is transferred into the liquid film through surface shear stress at the boundary between the liquid film and the substrate Theoretically, the contact line of the buffer sheet will be displaced from the point closest to the center of rotation to the point farthest from the point of rotation. However, at various angular speeds, instabilities will be created at the contact line and at the free surface of the liquid film and air. These instabilities cause non-uniform residence times and drying rates resulting in non-uniform spatial distribution of solute. FIG. 2 illustrates such a non-uniform distribution of a green reporter dye after drying of a solution of this dye using a centrifuge (only right half of the slide was wetted and dried to provide a reference).

The squeegee method also suffers from the previously described drying problems. Theoretically, the tip of the squeegee is not in contact with the glass surface because a thin layer of solution acts as a lubricant. However, in practice, the presence of dust or a change in pressure applied along the squeegee lip may result in the formation of a streak of buffer, thus leaving residue after complete evaporation. FIG. 3 illustrates such a non-uniform distribution of a green reporter dye (streaks) after drying of a solution of this dye using a squeegee (only right half of the slide was wetted and dried to provide a reference).

Simple evaporation of the buffer may result in local non-uniformity because of various drying artifacts. Within a given feature, the buffer may dry from the outside towards the inside (depining), thus concentrating the precipitated salts in a location within the feature, or from the inside towards the outside (coffee ring effect), thus concentrating the precipitated salts on the feature edges. FIG. 4 shows examples of local non-uniformity after drying of dye solution by evaporation.

As such, the above-described drying protocols are each associated with various problems that can adversely impact the results obtained in a given array assay. Accordingly, there is a continued need for the development of new array assay protocols

### SUMMARY OF THE INVENTION

Ligand array assays and compositions for use in practicing the same are provided. A feature of the subject methods is that they include a wash step in which the ligand displaying surface of a sample exposed ligand array is washed with a an organic fluid, e.g., propylene carbonate. Also provided are kits for use in practicing the subject methods. The subject methods and kits find use in a variety of ligand array based applications, including genomic and proteomic applications.

### BRIEF DESCRIPTION OF THE FIGURES

FIG. 1 provides a view of an array dried using a prior art air knife drying method. Specifically, FIG. 1 shows the distribution of a green reporter dye after drying of a solution of this dye using a nitrogen gun. Only the right half of the slide was wetted and dried to provide a reference.

FIG. 2. Distribution of a green reporter dye after drying of a solution of the dye using a centrifuge. Only the right half of the slide was wetted and dried to provide a reference.

FIG. 3. Distribution of a green reporter dye (streaks) after drying of a solution of this dye using a squeegee. Only the right half of the slide was wetted and dried to provide a reference.

FIG. 4. Examples of local non-uniformity after drying of dye solution by evaporation.

FIG. 5 shows an exemplary substrate carrying an array, such as may be used in the devices of the subject invention.

FIG. 6 shows an enlarged view of a portion of FIG. 5 showing spots or features.

FIG. 7 is an enlarged view of a portion of the substrate of FIG. 6.

FIG. 8 shows the effect of the subject wash fluids on observed signals.

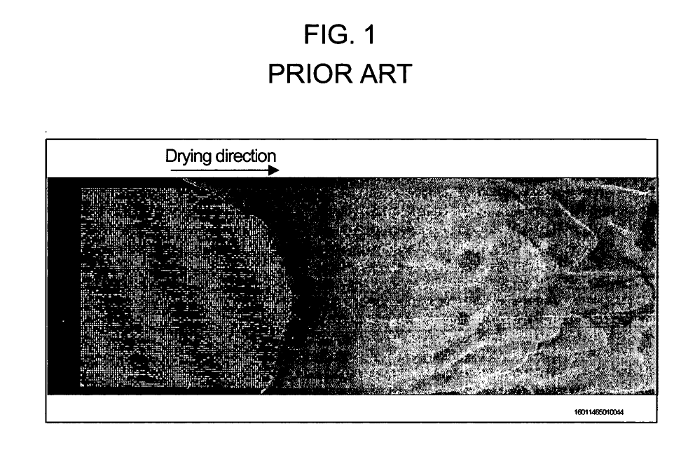

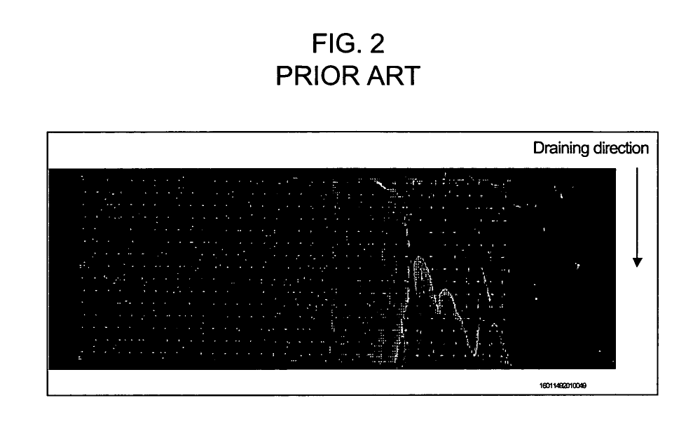

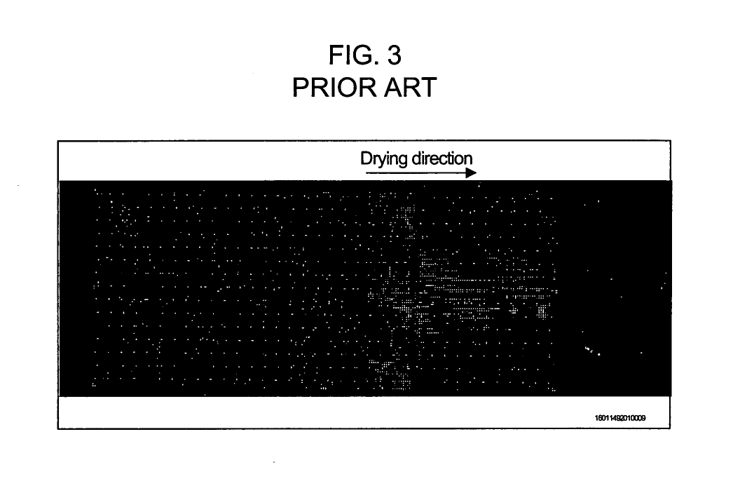

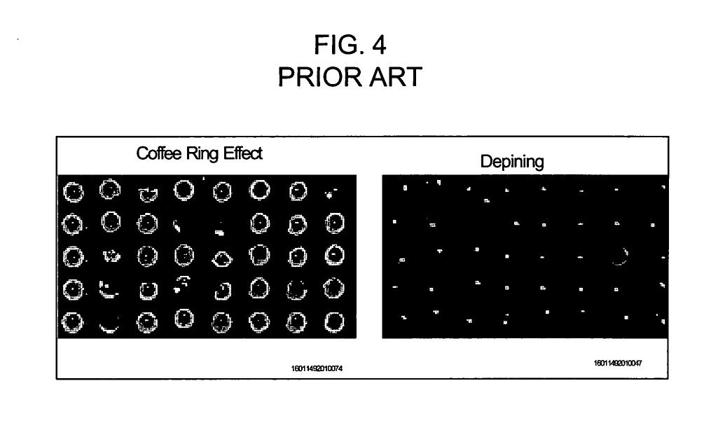

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

A “biopolymer” is a polymeric biomolecule of one or-more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups, one or both of which may have removable protecting groups).

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

With respect to methods in which premade probes are immobilized on a substrate surface, immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91-:5022-5026. The surface of a substrate may be treated with an organosilane coupling agent to functionalize the surface. One exemplary organosilane coupling agent is represented by the formula RₙSiY(₄₋ₙ) wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane (“GOPS”), the coupling chemistry of which is well-known in the art. See, e.g., Arkins, “Silane Coupling Agent Chemistry,” Petrarch Systems Register and Review, Eds. Anderson et al. (1987). Other examples of organosilane coupling agents are (y-aminopropyl)triethoxysilane and (y-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching probes to the support. For example, an oligonucleotide probe formed as described above may be provided with a 5′-terminal amino group that can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

In situ prepared ligand arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water. “Contact angle” of a liquid with a surface is the acute angle measured between the edge of a drop of liquid on that surface and the surface. Contact angle measurements are well known and can be obtained by various instruments such as an FTA200 available from First Ten Angstroms, Portsmouth, Va., U.S.A. Surfaces which are more hydrophobic (which have a lower surface energy) will have higher contact angles with water or aqueous liquids than surfaces which are less hydrophobic (and therefore a higher surface energy) (for example, a hydrophobic surface may have a water drop contact angle of more than 50 degrees, or even more than 90 degrees). The contact angle of an array (sometimes referenced as the “average contact angle” or “effective contact angle”) is the average contact angle of the features of that array and the inter-feature areas. Contact angles are measured with water unless otherwise indicated.

In certain embodiments, high surface energy regions, e.g., features, may have contact anglesthat are less than 45, less than 20 degrees (or less than 15, 10, or 5 degrees), while low surface energy, e.g., inter-feature, areas may have contact angles greater than 80 degrees (or even greater than 90, 95, 100, 105, 110, 115, 120 or 130 degrees).

Also, instead of drop deposition methods, light directed fabrication methods may be used, as are known in the art. Inter-feature areas need not be present particularly when the arrays are made by light directed synthesis protocols.

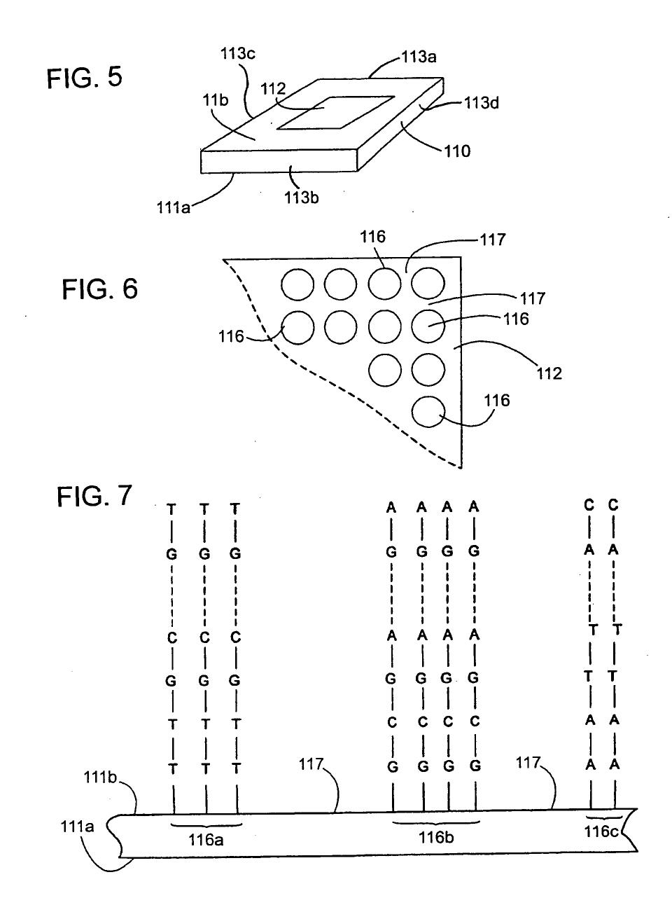

An exemplary array is shown in FIGS. 5-7,where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas that lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the-scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCI, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCI, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCI at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “sample” as used herein relates to a material or mixture of materials, typically, although not necessarily, in fluid form, containing one or more components of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DESCRIPTION OF THE SPECIFIC EMBODIMENTS

Ligand array assays and compositions for use in practicing the same are provided. A feature of the subject methods is that they include a wash step in which the ligand displaying surface of a sample exposed ligand array is washed with an organic fluid, e.g., propylene carbonate. Also provided are kits for use in practicing the subject methods. The subject methods and kits find use in a variety of ligand array based applications, including genomic and proteomic applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

Introduction

As summarized above, the subject invention provides methods and kits for performing array-based assays, i.e., array binding assays. The subject invention can be used with a number of different types of arrays in which a plurality of distinct polymeric binding agents (i.e., of differing sequence) are stably associated with at least one surface of a substrate or solid support. The polymeric binding agents may vary widely, however polymeric binding agents of particular interest include peptides, proteins, nucleic acids, polysaccharides, synthetic mimetics of such biopolymeric binding agents, etc. In many embodiments of interest, the biopolymeric arrays are arrays of nucleic acids, including oligonucleotides, polynucleotides, cDNAs, mRNAs, synthetic mimetics thereof, and the like.

While the subject methods and devices find use in array hybridization assays, the subject devices also find use in any suitable binding assay in which members of a specific binding pair, e.g., a ligand and receptor, interact. That is, any of a number of different binding assays may be performed with the subject methods, where typically a first member of a binding pair, typically referred to herein as the ligand, is stably associated with the surface of a substrate and a second member of a binding pair, typically referred to herein as the receptor, is free in a sample, where the binding members may be: antibodies and antigens, complementary nucleic acids, and the like. For ease of description only, the subject methods and devices described below will be described primarily in reference to hybridization assays, where such examples are not intended to limit the scope of the invention. It will be appreciated by those of skill in the art that the subject devices and methods may be employed for use with other binding assays as well, such as immunoassays, proteomic assays, etc.

In further describing the subject invention, the subject methods are described first in greater detail, followed by a review of representative applications in which the subject methods find use, as well as a review of representative systems and kits that find use in practicing the subject methods.

Methods

As summarized above, methods are provided for performing an array-based assay such as a hybridization assay or any other analogous binding interaction assay. A feature of the present methods is that a wash step that employs an organic wash fluid, as described in greater detail below, is employed. Accordingly, the subject methods differ significantly from prior art protocols in which such a wash step with an organic fluid is not performed.

In practicing the subject methods, the first step is typically to contact a sample, which in many embodiments is at least suspected to have (if not known to include) an analyte of interest, with an array of binding agents that includes a binding agent (ligand) specific for the analyte of interest. Contact of the sample and array occurs under conditions sufficient for the analyte, if present, to bind to its respective binding pair member that is present on the array. Thus, if the analyte of interest is present in the sample, it binds to the array at the site of its complementary binding member and a complex is formed on the array surface. Depending on the nature of the analyte(s), the array may vary greatly, where representative arrays are reviewed in the Definitions section, above. Of particular interest are nucleic acid arrays, where in situ prepared nucleic acid array are employed in many embodiments of the subject invention.

To contact the sample with the array, the array and sample are brought together in a manner sufficient so that the sample contacts the surface immobilized ligands of the array. As such, the array may be placed on top of the sample, the sample may be placed, e.g., deposited on the array surface, the array may be immersed in the sample, etc.

Following contact of the array and the sample, the resultant sample contacted or exposed array is then maintained under conditions sufficient and for a sufficient period of time for any binding complexes between members of specific binding pairs to occur. In many embodiments, the duration of this step is at least about 10 min long, often at least about 20 min long, and may be as long as 30 min or longer, but often does not exceed about 72 hours. The sample/array structure is typically maintained at a temperature ranging from about 40 to about 80, such as from about 40 to 70° C. Where desired, the sample may be agitated to ensure contact of the sample with the array.

In the case of hybridization assays, the substrate supported sample is contacted with the array under stringent hybridization conditions, whereby complexes are formed between target nucleic acids that are complementary to probe sequences attached to the array surface, i.e., duplex nucleic acids are formed on the surface of the substrate by the interaction of the probe nucleic acid and its complement target nucleic acid present in the sample. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCI, 15 mM trisodium citrate), 50 mM sodium phosphate (pH7.6), 5×Denhardt's solution, 10% dextran sulfate, followed by washing the filters in 0.1×SSC at about 65° C. Hybridization involving nucleic acids generally takes from about 30 minutes to about 24 hours, but may vary as required. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

Once the incubation step is complete, the array is typically washed at least one time to remove any unbound and non-specifically bound sample from the substrate, generally at least two wash cycles are used. Washing agents used in array assays are known in the art and, of course, may vary depending on the particular binding pair used in the particular assay. For example, in those embodiments employing nucleic acid hybridization, washing agents of interest include, but are not limited to, salt solutions such as sodium, sodium phosphate and sodium, sodium chloride and the like as is known in the art, at different concentrations and may include some surfactant as well. Such wash solutions are water-based wash solutions, e.g., they are aqueous solutions.

As mentioned above, a feature of the subject invention is that the methods include at least one washing step in which the array surface is washed with an organic fluid. By organic fluid is meant a fluid, typically solvent, that is made up of carbon containing molecules. In many embodiments, this particular wash step is the last wash step performed prior to the array reading step, described below. This final wash step provides a number of benefits, which benefits are reviewed in greater detail below.

In certain embodiments, the organic wash fluid employed in the wash step is a high surface tension fluid. As such, the surface tension of the fluid employed in this wash step typically exceeds at least about 40, and in certain embodiments exceeds at least about 42, including at least about 45 mN/m (as measured at 25° C.). (The determination of a given fluid's surface tension is performed by well-known and standard procedures, and may also be made by referring to a reference source that provides the surface tension of various fluids at various temperatures)

Another feature of the organic wash fluid that is employed in many embodiments of the subject invention is that the fluid has a low vapor pressure. As such, the fluid typically has a vapor pressure that is less than about 10⁻¹ KPa, usually less than about 10⁻²KPa and more usually less than about 10⁻³ KPa at standard temperature and pressure conditions i.e., STP conditions (0° C.; 1 ATM). (The determination of a given fluid's vapor pressure is performed by well-known and standard procedures, and may also be made by referring to a reference source that provides the vapor pressure of various fluids under various conditions)

Furthermore, in certain embodiments the fluid has a high viscosity. In such embodiments the viscosity of the fluid typically exceeds about 1.2, and in certain embodiments exceeds about 2, such as about 2.5 cP (as measured at 25° C.).

The non-dimensional capillary number of the fluid should be in the range of from about 10⁻² to about 10⁻⁶. The capillary number Ca is defined as Ca=(μ.U)σ, where μ is the viscosity, U is the linear speed and σ is the surface tension. This number provides a range within which the slide drag-out speed can be adjusted to account for the particular fluid properties. However, while Ca serves as a coarse guide for controlling mechanical aspects of the flow, other subtleties such as the evaporation rate and fluid adherence to the substrate manifested in the disjoining pressure influence the motion of the contact line.

In many embodiments, the wash fluid is one that is miscible with the fluid that previously contacted the array surface in the particular protocol being performed, e.g., the sample or the previous wash fluid. As such, in many embodiments, the organic wash fluid is one that is miscible with aqueous fluids. For purposes of the present invention, a first and second fluid are considered to be miscible if the first fluid is soluble in the second fluid when the two fluids are present in a ratio of first to second fluid of at least 0.25/1, such as at least about 0.5/1, including at least about 0.75/1, such as at least about 1/1. In many embodiments, the organic wash fluid is one in which the analyte or ligands of the array, e.g., nucleic acids, is not soluble. In certain embodiments where the analyte and ligand therefore are nucleic acids, the organic fluid is not a nucleic acid solvent, by which is meant that nucleic acids, e.g., DNA, RNA, as well as mimetics thereof, are not soluble in the low surface tension fluid. In these embodiments, the solubility of nucleic acids in the fluid is described as the fraction of hybridized nucleic acid that are melted upon contact with the fluid (as measured at Standard Temperature and Pressure). This fraction does not exceed about 20%, (including about 15%, about 10%, about 5%) and typically does not exceed about 1%, e.g., over a given time period, such as a period of at least about 10 min, including at least about 60 min, including at least about 6 hours or longer.Specific organic wash fluids of interest include, but are not limited to: propylene carbonate; ethylene carbonate, benzophenone; benzyl cyanide, nitrobenzene, 2-phenylethanol, 1,3-propanediol, 1,4-butanediol, 1,5-pentanediol, diethyleneglycol, triethyleneglycol, glycerol, dimethyl sulfoxide (DMSO), N-methyl formamide, N-methyl pyrrolidone, and the like. Also of interest are the low surface tension organic wash fluids disclosed in Application Serial No.______(Agilent Docket No. 10030682-1) filed on even date herewith, the disclosure of which is herein incorporated by reference.

In certain embodiments, the organic wash fluid is one that does not include a cosolvent. In yet other embodiments, this wash fluid may include a cosolvent. When a cosolvent is present, the amount of the cosolvent typically will not exceed about 50% (v/v), such as about 20% (v/v). Representative cosolvents that may be present include, but are not limited to: acetonitrile, acetone, ethyl acetate, hexane, diethyl ether, methanol, ethanol, acetylacetone, diethylcarbonate, chloroform, methylene chloride, and the like. In the final wash step in which the organic wash fluid (as described above) is employed, the wash step may be performed using any convenient protocol. In many embodiments, this wash step includes immersing the array in a sufficient volume of the organic wash fluid and then removing the array from the wash fluid. While immersed, the array and/or wash fluid may be agitated as desired. In certain embodiments, the array may be removed from the wash fluid at a constant rate, e.g., at a rate of from about 0.01 cm/sec to about 10 cm/sec.

Following removal of array from the wash fluid as described above (so that excess fluid on the surface is removed from the array surface) the surface is then typically dried, e.g., by using an evaporation protocol in which remaining wash fluid on the surface of the array is allowed to evaporate. As such, the surface of the array is typically maintained in an environment that allows for evaporation of the remaining solvent, such as at a temperature of from about 0 to about 100, including from about 20 to about 50° C. The atmosphere during drying may be air, or a suitable anhydrous atmosphere, e.g., dry nitrogen gas, argon, helium and the like. Conveniently, drying of the array surface as described above may be carried out in a closed system, e.g., chamber, that provides for control of the temperature and atmosphere to provide for the desired conditions. This drying step may take from about 0.01 to about 30 min, including from about 1 to about 5 min.

Following the above array/sample contact step and wash step, the presence of any resultant binding complexes on the array surface is then detected, e.g., through use of a signal production system, e.g., an isotopic or fluorescent label present on the analyte, etc. In other words, the resultant dried array is then interrogated or read to detect the presence of any binding complexes on the surface thereof, e.g., the label is detected using colorimetric, fluorimetric, chemiluminescent or bioluminescent means. The presence of the analyte in the sample is then deduced or determined from the detection of binding complexes on the substrate surface.

The organic fluid wash step, as described above, may be incorporated into an automated array processing, e.g., assaying protocol, in which one or more of the individual steps of the protocol, including the subject wash step, are performed using automated machinery or instruments.

Utility

The methods of the present invention find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Where the arrays are arrays of polypeptide binding agents, e.g., protein arrays, specific applications of interest include analyte detection/proteomics applications, including those described in: 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read, following the subject wash in an organic fluid. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample, or whether an organism from which the sample was obtained exhibits a particular condition, for example, cancer). The results of the reading (processed or not) may be forwarded (such as by communication) to a remote location if desired, and received there for further use (such as further processing).

Kits

Kits for use in analyte detection assays, as described above, are also provided. The kits at least include an organic wash fluid, as described above. The kits may further include one or more additional components necessary for carrying out an analyte detection assay, such as one or more ligand arrays, sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include buffers (such as hybridization buffers), wash mediums, enzyme substrates, reagents for generating a labeled target sample such as a labeled target nucleic acid sample, negative and positive controls and written instructions for using the array assay devices for carrying out an array based assay.

Such kits also typically include instructions for use in practicing array-based assays according to the subject invention where a wash step employing an organic wash fluid is performed. The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

### Experimental

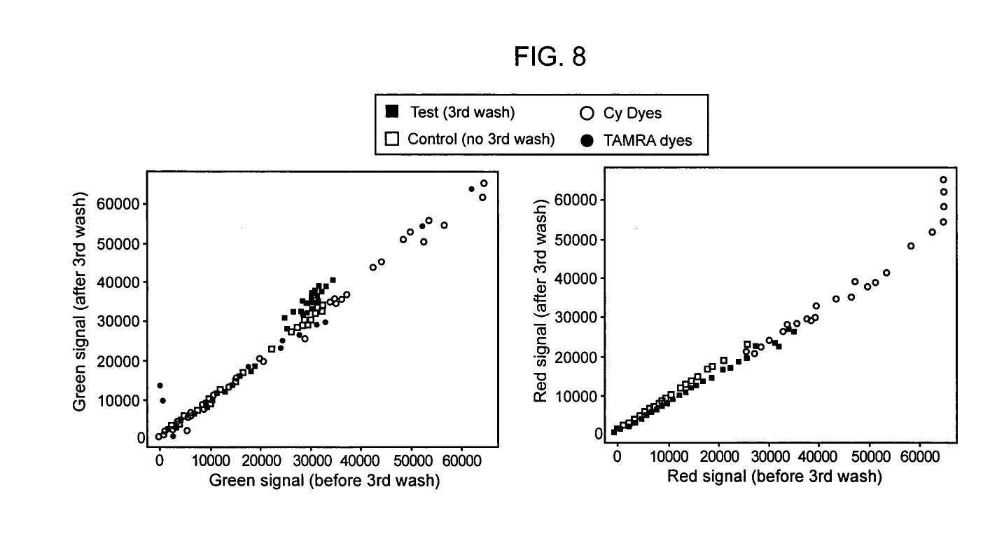

The processing steps of in situ microarrays at customer sites consist of hybridization, washings, drying and scanning. Using this invention, an Agilent in situ Human catalog array (part #G411 OA) (Agilent Technologies, Palo Alto, Calif.) was hybridized to a sample of 1.5 μg Cy3/Cy5 labeled RNA (Cy3 channel was MG63 cell line and Cy5 channel was brain) and washed using the current recommended protocols, as described in Agilent Publication Number G4140-90010. Per the protocol, at the end of the 2^{nd} wash, the array was coated with a sheet of 0.06×SSC buffer and 0.05% Triton-X 102 as surfactant. Instead of performing the standard drying process following these washes, the array was then transferred to a 3^{rd} propylene carbonate wash solution. After agitation of the solution, the array was then removed from the solution at a constant speed. The above action resulted in the formation of a droplet of propylene carbonate on each feature of the array, but little if any propylene carbonate in the interfeature areas. The resultant array was then dried in an air atmosphere at a temperature of 25° C. for approximately 20 min. The resultant slide was then scanned and the data processed per the protocol described in Agilent Publication 5988-5022. FIG. 8 shows the effect of a third wash on the signals (no change).

It is evident from the above results and discussion that embodiments of the above-described invention may provide a useful method of performing array-based assays. Examples of one or more benefits which may be obtained in different emobidments follow. Employing a wash step according to the present invention may solve one or more of the problems experienced when other protocols are employed, such as problems associated with lack of local uniformity, lack of global uniformity and lack of reproducibility. Employing an organic wash according to the present invention can remove all the salts from the wash buffer(s) previous to drying. Therefore, even if evaporation gradients occur, no particle is deposited on the array surface upon drying of the wash solution. Consequently, no scanning artifacts or local modification of the dye quantum yields can occur. Furthermore, an organic fluid wash according to the present invention does not affect the binding of the targets with the probes attached on the surface because a wash fluid is employed in which single stranded nucleic acids are not soluble. Therefore, during drying of the organic wash fluid, no stringency artifacts are created and no local denaturation occurs. In addition, the invention is applicable evenly to the array resulting in an excellent global drying uniformity and high reproducibility. The invention does not require special equipment such as centrifuges or nitrogen guns, thus facilitating its deployment. Finally, the methodology is easily automated, and may be incorporated into an overall automated array processing system. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of determining whether an analyte is present in a sample, said method comprising:
(a) contacting said sample with a surface of a substrate having immobilized thereon a ligand that specifically binds to said analyte;
(b) washing said surface with a an organic wash fluid in which said analyte and ligand therefore are not soluble; and
(c) detecting any resultant binding complexes on said surface to determine whether said analyte is present in said sample.

2. The method according to claim 1, wherein said fluid is a high surface tension fluid.

3. The method according to claim 1, wherein said wash fluid has a surface tension that is at least about 40 mN/m.

4. The method according to claim 1, wherein said wash fluid has a vapor pressure of less than about 10⁻¹ KPa.

5. The method according to claim 4, wherein said wash fluid comprises propylene carbonate.

6. The method according to claim 1, wherein said analyte is a nucleic acid.

7. The method according to claim 1, wherein said ligand is a nucleic acid.

8. The method according to claim 7, wherein said substrate displaying said nucleic acid ligand is a nucleic acid array.

9. The method according to claim 8, wherein said nucleic acid array is an in situ prepared nucleic acid array.

10. The method according to claim 1, wherein said method further comprises at least one additional wash step prior to said wash step (b).

11. The method according to claim 10, wherein said at least one additional wash step comprises washing said substrate surface with an aqueous fluid.

12. The method according to claim 11, wherein said organic wash fluid is miscible with said aqueous fluid.

13. The method according to claim 1, wherein said method is a method of assaying said sample for the presence of two or more distinct analytes.

14. The method according to claim 1, wherein at least a portion of said method is automated.

15. A method comprising transmitting data representing a result obtained by the method according to claim 1, from a first location to a second location.

16. A method according to claim 15, wherein said second location is a remote location.

17. A method comprising receiving data representing a result of a method of claim 1.

18. A kit for performing an assay, said kit comprising:
(a) a high surface tension organic fluid in which nucleic acids are not soluble; and
(b) instructions for using said wash fluid in a method according to claim 1.

19. The kit according to claim 18, wherein said wash fluid has a surface tension that is at least about 40 mN/m.

20. The kit according to claim 18, wherein said wash fluid has a vapor pressure of less than about 10⁻¹ KPa.

21. The kit according to claim 20, wherein said wash fluid comprises propylene carbonate.

22. The kit according to claim 18, wherein said wash fluid comprises at least about 2% (v/v) of a cosolvent.

23. The kit according to claim 18, wherein said kit further comprises an array comprising at least two different ligands.

24. The kit according to claim 23, wherein said ligands are nucleic acid ligands.

25. A substrate produced according to the method of claim 1.

26. The substrate according to claim 25, wherein said substrate is a substrate of a ligand array having two or more distinct ligands immobilized on a surface of said substrate.

27. The substrate according to claim 26, wherein said ligand array is a nucleic acid array.

28. The substrate according to claim 27, wherein said nucleic acid array is an in situ prepared nucleic acid array.
